(19) United States
(12) Patent Application Publication
(10) Pub. No.: US 2006/0064051 A1
(43) Pub. Date: Mar. 23, 2006

(54) Multiple port catheter connector

(76) Inventor: James R. Gross, Richmond, IL (US)

Correspondence Address: Kimberly Diliberti;Paralegal, 1430 Waukegan Road, McGaw Park, IL, 60085, US

(21) Appl. No.: 11/231,665

(22) Filed: Sep. 21, 2005

Related U.S. Application Data
(63) Continuation of application No. 10/389,621, filed on Mar. 14, 2003, now abandoned.

(51) Int. Cl. A61N 1/30

(52) U.S. Cl. 604/19; 604/246

(57) ABSTRACT

The invention described herein relates to a multiple port catheter or medical tubing connector comprising a generally elongate central housing portion having a closed first end and open second end; a central lumen running along a longitudinal axis within said housing portion, said central lumen terminating at an open second end of the housing portion; a plurality of individual side ports extending from said central housing portion, each side port having an open end in communication with a port lumen within, and each port lumen individually converging in communication with said central lumen; and a transparent planar viewing window located on the side of said housing portion. The multiple port catheter connector can be used in a variety of scenarios within the medical field which require coordination and attachment of a plurality of catheters to conjoin into a unified efflux or exit flow, such as multiple site drainage or patients requiring multiple intravenous administration.

### FIELD OF THE INVENTION

The invention relates to the field of medical devices. In particular, the invention pertains to devices for connecting catheters.

### BACKGROUND OF THE INVENTION

Multiple port catheter or medical tubing connectors are well-known in the art. A variety of catheter connectors which permits connection of two or more tubes or catheters to one another are available. Examples of such devices include the BLAKE™ CARDIOCONNECTOR 2:1 (available from Ethicon, Inc., Somerville, N.J.) and that described in Rafelson U.S. Pat. No. 4,662,871, both of which include “Y-shaped” catheter connectors. Another multiple catheter connector is described in Abramson U.S. Pat. No. 4,508,533.

One problem associated with prior catheter connectors is their specificity to particular equipment, such as the number and sizes of catheters to be attached. Thus, the connectors contain the structural features and dimensions which limit their adaptability to circumstance and flexibility of use. In other words, some connectors are designed as one or two or three tube connectors thereby limiting their use to one, two or three tube attachments, respectively. This limits the user to a predetermined number of tubes that may be used in a surgical procedure, for example, because the number of tubes to be attached must be established prior to the procedure or using the connector.

Another problem associated with catheter connectors is the awkwardness of using separate secondary components, such as separate detached end caps. Yet another problem associated with previous catheter connectors is the ability to monitor or view the function of the connector, i.e., whether flow is occurring without blockage or obstruction and the ability to clearly distinguish between each individual port's function. Because of their overall symmetrical configurations, and in come cases the opacity of the connector material, confusion between each port and its associated tube attached can result.

There is a need in the medical device field for improved tubing or catheter connectors. There is a need for a multiple port tubing or catheter connector that can be used in a variety of circumstances which require coordination and attachment of a plurality of catheters, permits selective use of connector features, and which afford the user an improved ability to monitor its operation.

### SUMMARY OF THE INVENTION

The invention provides an improved multiple port catheter connector that is easy to use, adaptable, and affords the user advantages associated with its use. The benefits associated with the multiple port catheter connector of the invention are derived from the combination of its structural and functional features. It has been discovered that a catheter connector can be constructed to improve the ability to monitor its operation, to facilitate its positioning, to increase the flexibility of its use by accommodating differently sized catheters and tubing and permitting selective use of its features, to minimize the number of separate components, to reduce the need for anticipating its suitability relative to secondary equipment, to facilitate attachment of catheters ot other tubing, to distinguish between or among the side ports and their operation relative to one another, and to be relatively easy to manufacture.

The invention provides a multiple port catheter connector comprising:
a generally elongate central housing portion having a closed first end and open second end;
a central lumen running along a longitudinal axis within said housing portion, said central lumen terminating at an open second end of the housing portion;
a plurality of individual side ports extending from said central housing portion, each side port having an open end in communication with a port lumen within, and each port lumen individually converging in communication with said central lumen; and
a transparent planar viewing window located on the side of said housing portion.

Preferably, the transparent viewing window is elongated and permits generally undistorted viewing of the interior of the central lumen, thereby revealing the converging juncture of the central lumen with each of the side port lumens. Accordingly, the relationship of each port and the central lumen can be readily distinguished and inspected for flow or obstruction.

The invention also provides a multiple port catheter connector comprising:
a generally elongate central housing portion having a closed first end and open second end;
a central lumen running along a longitudinal axis within said housing portion, said central lumen terminating at an open second end of the housing portion;
a plurality of individual side ports extending from said central housing portion, each side port having an open end in communication with a port lumen within, and each port lumen individually converging in communication with said central lumen;
a transparent planar viewing window located on the side of said housing portion; and wherein said closed first end further comprises an attachment structure for securing the multiple port catheter connector to an object.

The attachment structure permits more stabilized affixation of the multiple port catheter connector to another object, such as the side of a bed or part of a stand, such that catheter connector is not merely suspended without restraint by the catheters to which it is attached.

The invention further provides a multiple port catheter connector comprising:
a generally elongate central housing portion having a closed first end and open second end;
a central lumen running along a longitudinal axis within said housing portion, said central lumen terminating at an open second end of the housing portion;
a plurality of individual side ports extending from said central housing portion, each side port having an open end in communication with a port lumen within, and each port lumen individually converging in communication with said central lumen;
wherein each of said individual side ports comprises a beveled tip; and
a transparent planar viewing window located on the side of said housing portion.

The beveled tip of the side ports facilitates the over-fitting of a catheter onto the end of the side port. The beveled or angled configuration of the side port tip provides a gradual introduction of the tip into the catheter. Preferably, the edges of the side port tip are smooth and rounded. In a preferred embodiment of the invention, all but one of the side ports of the multiple port connector of the invention is closed and sealed by a removably attachable tethered end cap, thereby affording the user various options of catheter or tubing attachment sites and number upon presentation of a single unit of the device.

The multiple port catheter connector can be used in a variety of scenarios within the medical field which require coordination and attachment of a plurality of catheters to conjoin into a unified efflux or exit flow. The device of the invention is particularly useful with multiple site drainage, or patients requiring multiple intravenous administration. These and other advantages associated with the invention will become apparent from the following text.

### BRIEF DESCRIPTION OF THE DRAWINGS

The following figures further illustrate the features of the invention:

FIG. 1 is a side view of the multiple port catheter connector according to one embodiment of the invention.

FIG. 2 is a side view of the multiple port catheter connector showing the attachment element and a port end cap in released positions according to one embodiment of the invention.

FIG. 3 is a side view of the multiple port catheter connector with four catheters coupled to the device according to one embodiment of the invention.

FIG. 4 is an angled side view of the multiple port catheter connector according to one embodiment of the invention.

FIG. 5 is a cross-sectional view showing the interior of the multiple port catheter connector according to one embodiment of the invention.

### DETAILED DESCRIPTION OF THE INVENTION

As used herein, the term “catheter” is meant to encompass all varieties and forms of medical tubing and the like that can benefit from attachment and use in association with the connector of the invention.

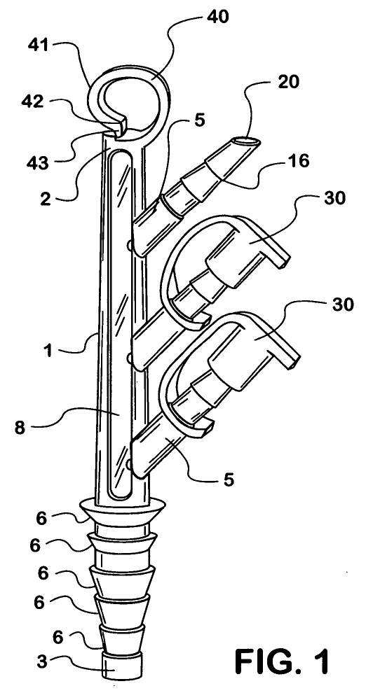

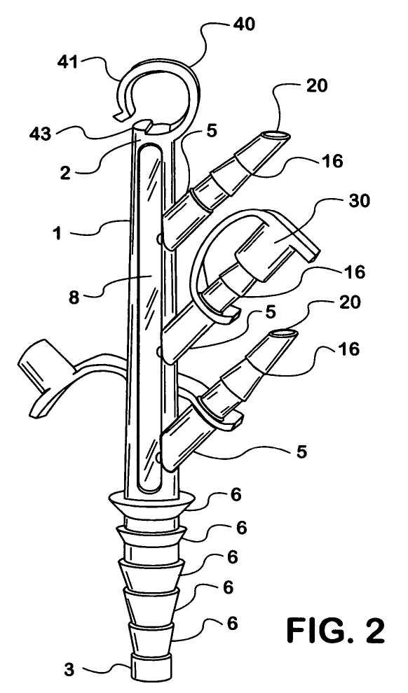

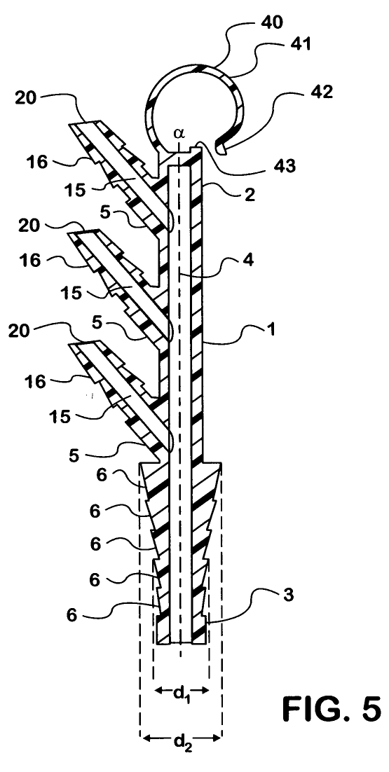

Referring to FIGS. 1, 2, 3, 4, and 5, the multiple port catheter connector of the invention comprises a generally elongate central housing portion 1 having a closed first end 2 and an open second end 3, and a central lumen 4 (see FIG. 5) running along the longitudinal axis α (alpha) within the central housing portion 1. The central lumen 4 is a common confluent lumen of the side ports and their respective lumens, and terminates at the second end 3 of the housing portion.

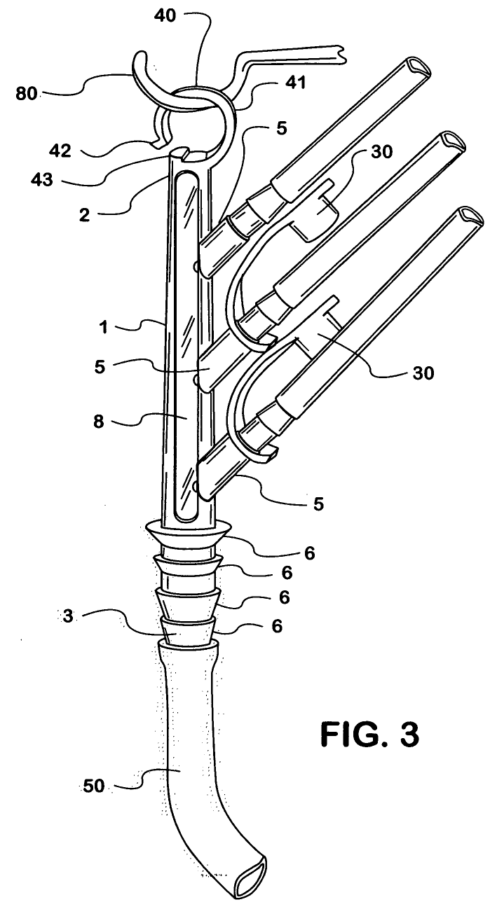

The second end 3 of the central housing portion 1 is structured for attachment of the exit or efflux catheter 50 (see FIG. 3) to the connector of the invention. Furthermore, the second end 3 of the housing portion 1 is preferably a “universal” structure that can accommodate various catheter sizes and dimensions and permit the secure attachment thereof onto the connector. Thus, the second end 3 of the housing portion 1 can comprise a series of annular tapered steps 6 which are arranged in a sequential gradient with respect to diameter. The diameter range of the annular tapered steps can vary, but typically the diameter range can be selected to accommodate tubing internal diameters from about 10 mm to about 15 mm. Referring now to FIG. 5, the tapered step having the largest diameter d₂ is located nearest the first end 1 of the housing, whereas the tapered step having the smallest diameter d₁ is located adjacent or proximal to the opening of the second end 3. In use, the exit catheter 50 can be superimposed over the second end 3 and advanced until the end of the catheter fits over the annular tapered step that provides the optimal secure fit for the catheter, such as depicted in FIG. 3.

Extending from the side of the central housing portion 1 is a plurality of individual side ports 5 located at different positions along the length of the central housing portion 1. The elongate configuration of the central housing portion 1 is important because together with the transparent planar viewing window 8 (discussed more below) and side port locations, the user is afforded the ability to readily distinguish each of the side ports 5 from one another as well as monitor the individual flow of each side port into the central lumen along a substantial extent of the length of the central lumen. This feature of the invention is important when the multiport catheter connector of the invention is used to transport bodily fluids, which can be high viscosity and/or the tendency to obstruct, clump or clot. Such blockage or flow inhibition can result in stagnation or backflow of fluid.

The transparent planar viewing window 8 is an important feature of the invention. This feature is significantly advantageous over the mere use of transparent or translucent materials, which can nevertheless provide a distorted image of that which is within or on the opposite side of the material. It is therefore significant that the viewing window possess at least a planar exterior surface so as to reduce such distortion from occurring and provide a more precise and accurate image of the interior lumens of the multiport catheter connector. The number and position of transparent planar viewing window(s) can vary. It is preferred, however, that the transparent planar viewing window 8 have dimensions, i.e., width and length, sufficient to permit observation of a significant amount of the central lumen 4. At least one viewing window 8 should be positioned so as to permit viewing of the juncture of the side port lumens 15 (see FIG. 5) converging into the central lumen 4 to optimize the benefit of the viewing window.

The number and orientation of the side ports 5 can vary, provided the abovementioned functionality is not significantly compromised. With respect to number, the multiport catheter connector of the invention comprises at least two side ports. For example, the multiport catheter connector of the invention can contain three or four side ports. In order to enhance the flexibility of its use, however, it is preferred that there are at least three side ports. With respect to orientation, the side ports can be aligned as deppicted in the figures or, alternatively, staggered along the length of the central housing portion. Again, it is preferred that there is enough space between the side ports from one another to permit distinction among them when viewing their flow.

Referring to FIGS. 1, 2, 4 and 5, each of the side ports 5 comprises at least one annular tapered step 16. As in the case of the second end 3 of the central housing portion 1, each side port 5 can likewise contain a series of sequential annular tapered steps or barbs arranged in a gradient according to diameter to permit an optimal fit of the catheter over which is attached. The diameter of the side port annular tapered steps can vary. Typically, the first annular step of the side port can have a diameter structured to accommodate a tubing internal diameter of about 5 mm. Preferably, the tip 20 of each side port 5 is beveled or angled. The beveled tips 20 of the side ports 5 facilitate the overfitting of a catheter onto the end of the side port 5. The beveled or angled configuration of the side port tip provides a gradual introduction of the tip 20 into the catheter. Even further preferred are edges of the beveled side port tip 20 that are smooth and rounded.

As can be seen from FIGS. 1 through 3, all but one of the side ports preferably has an attached tethered end cap 30 associated with it, so that the end caps themselves are not a separate component from the connector. Since the device is used to couple at least two catheters, one of the side ports and the second end of the housing need not include a tethered end cap, as shown in the Figures. The unused side ports, however, preferably comprise a tethered end cap 30 because this affords the user of the connector to select the number of side ports 5 to be used from a variety of options in number and attachment site. In this respect, the multiport connector of the invention provides the user with flexibility of use upon presentation of a single unit of the device. Accordingly, the user does not need to pre-select from among a collection or array of particular connectors which are specifically structured according to the anticipated number of catheters to be coupled. Furthermore, the user does not need to pre-calculate or estimate the number of catheters or tubing to be attached in advance of the procedure, for example.

Each tethered end cap 30 is preferably structured to securely fit onto the end of the side port 5 to which it is associated. To reduce the likelihood of leakage and minimize environmental exposure, the tethered end cap is structured to interact with the annular tapered step 16 of the side port 5 so as to resist unintentional detachment of the tethered end cap therefrom. In one embodiment (not shown), the interior wall of the end cap can include an annular extension which is structured to surround a step on the exterior of the side port to provide a tight fit. Alternatively, the inside of the end cap can further comprise a plug-like protrusion structured to extend down into the inside of the side port end, thereby providing two occlusion constructs when fitted over the end of the side port. The tethered end cap can be composed of a variety of sterilizable materials, such as plastic, rubber or elastomeric polymer. Preferably, the tethered end cap is composed of a resilient material.

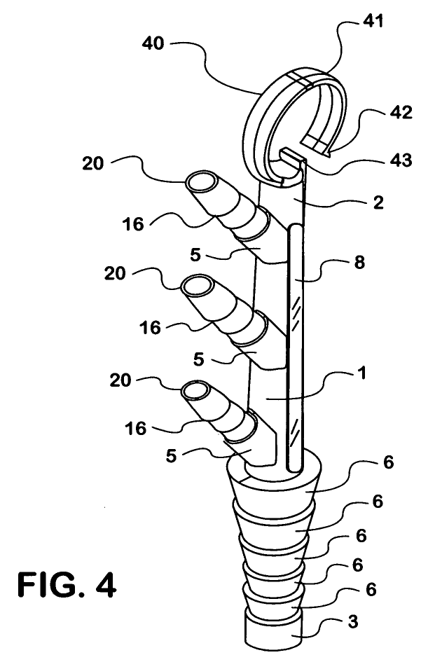

The multiple port catheter connector of the invention can further comprise an attachment structure 40 to secure the connector to another object 80 (see FIG. 3). The attachment structure 40 thereby permits more stabilized affixation of the multiple port catheter connector to another object, such as the side of a bed or part of a stand, such that catheter connector is not merely suspended without restraint by the catheters to which it is attached. The attachment structure 40 can also reduce the risk of entanglement of the connector with other equipment in the vicinity at the same time. In one embodiment, the attachment structure 40 is in the form of a loop 41 as shown in the figures. As can be seen in FIG. 4, one loop structure comprises a latch 42 which interacts with a corresponding locking step 43 at the top of the first end 2 of the central housing portion 1 to close the loop. In an alternative embodiment, the attachment structure 40 can be in the form of an open hook. Although a variety of attachment or fastening structures, both separate and integrated, can be used with the multiple port catheter connector of the invention, attachment structures which are integrally molded or attached to the connector are preferred in order to reduce separate components and for a ease of manufacture.

The multiple port catheter connector can be made using conventional techniques and equipment readily available to those skilled in the art. The device can be molded, shaped and finished using conventional molding equipment, for example. The multiple port catheter connector can be made from a variety of plastics and polymeric materials, provided the viewing window is composed of a material which is transparent and permits generally undistorted viewing of the central lumen within the housing portion. Preferably and for ease of manufacture, the entire device other than tethered end caps if present, is composed of the same material. Although composed of the same material, the surface of the device, other than the viewing window, can be modified. INDUSTRIAL APPLICABILITY

The multiple port catheter connector can be used in a variety of scenarios within the medical field which require coordination and attachment of a plurality of catheters to conjoin into a unified efflux or exit flow. For example, the device of the invention can be used in patients requiring multiple intravenous administration. The multiple port catheter connector of the invention affords the user enhanced control and flexibility of use. By virtue of its construction and structural features, the user can simplify the task of connecting and separating catheters. More specifically, the device of the invention permits selective use of the number of side ports, generally undistorted viewing of the interior central lumen and fluid flow within, and “universal” attachment capability with respect to catheter sizes. Furthermore, the multiple port catheter connector of the invention is relatively easy to manufacture.

The invention has been described herein above with reference to various and specific embodiments and techniques. It will be understood by one of ordinary skill in the art that reasonable modifications and variations of such embodiments and techniques are possible without significantly departing from the spirit or scope of the invention as defined by the claims below.

1. A medical multiple port catheter connector structured for attachment of medical tubing said connector comprising:
a generally elongated central housing portion having a closed first end and open second end;
a central lumen running along a longitudinal axis within said housing portion, said central lumen terminating at an open second end of the housing portion;
a plurality of individual side ports extending from said central housing portion, each side port having an open end in communication with a port lumen within, and each port lumen individually converging in communication with said central lumen; and
an elongated transparent planar viewing window located on the long side of said housing portion permitting generally undistorted viewing of the interior of said central lumen and converging juncture thereof with each of said port lumen.

2. The multiple port catheter connector according to claim 1, wherein said second end of said housing portion comprises annular tapered steps on the exterior surface.

3. The multiple port catheter connector according to claim 2, wherein said annular tapered steps are sequentially arranged in a gradient of diameters such that a largest diameter annular step is located nearest the first end of the housing and a smallest diameter annular step is located proximal to the open second end.

4. The multiple side port catheter connector according to claim 1, wherein each of said side ports comprises a beveled tip.

5. The multiple port catheter connector according to claim 1, wherein each of said side ports comprises at least one annular tapered step on the exterior.

6. The multiple port catheter connector according to claim 1, wherein at least one of said side ports further comprises a tethered end cap attached to said side port.

7. The multiple port catheter connector according to claim 5, wherein at least one of said side ports further comprises a tethered end cap attached to said side port, and said end cap is structured to be removably attached over said annular tapered step of said side port so as to sealably cap said side port and resist detachment therefrom.

8. The multiple port catheter connector according to claim 7 wherein all but one the side ports are capped.

9. The multiple port catheter connector according to claim 6, wherein the tethered end cap is composed of a resilient polymeric material.

10. The multiple port catheter connector according to claim 1 comprising two individual side ports.

11. The multiple port catheter connector according to claim 1 comprising three individual side ports.

12. The multiple port catheter connector according to claim 1 comprising four individual side ports.

13. A medical multiple port catheter connector structured for attachment of medical tubing said connector comprising:
a generally elongated central housing portion having a closed first end and open second end;
a unitary central lumen running along a longitudinal axis within said housing portion, said central lumen terminating at an open second end of the housing portion;
a plurality of individual side ports extending from said central housing portion, each side port having an open end in communication with a port lumen within, said port lumen converging in communication with said central lumen;
a transparent planar viewing window located on the long side of said housing portion permitting generally undistorted viewing of the interior of said unitary central lumen and converging juncture thereof with each of said port lumen;
and wherein said closed first end further comprises an attachment structure for securing the multiple port catheter connector onto an object.

14. The multiple port catheter connector according to claim 13 wherein said attachment structure is an integral structure of the housing.

15. The multiple port catheter connector according to claim 13 wherein said attachment structure is in the form of an adjustable loop.

16. The multiple port catheter connector according to claim 13 wherein said attachment structure is in the form of a hook

17. A medical multiple port catheter connector structured for attachment of medical tubing, said connector comprising:
a generally elongated central housing portion having a closed first end and open second end;
a central lumen running along a longitudinal axis within said housing portion; said central lumen terminating at an open second end of the housing portion;
a plurality of individual side ports extending from said central housing portion, each side port having an open end in communication with a port lumen within, said port lumen converging in communication with said central lumen;
wherein each of said individual side ports comprises a beveled tip; and
a transparent planar viewing window located on the long side of said housing portion permitting generally undistorted viewing of the interior of said central lumen and converging juncture thereof with each of said port lumen.

18. (canceled)
